(19) United States
(12) Patent Application Publication
(10) Pub. No.: US 2006/0099646 A1
(43) Pub. Date: May 11, 2006

(54) Bret assay

(76) Inventor: Anders Heding, Copenhagen (DK)

Correspondence Address: EDWARDS & ANGELL, LLP, P.O. BOX 55874, BOSTON, MA, 02205, US

(21) Appl. No.: 10/531,072

(22) PCT Filed: Oct. 10, 2003
(86) PCT No.: PCT/DK03/00687

§ 371 Date: Sep. 30, 2005

(30) Foreign Application Priority Data
Oct. 11, 2002 (DK) PA 2002 01568
Jan. 22, 2003 (DK) PA 2003 00076

(51) Int. Cl. G01N 33/53

(52) U.S. Cl. 435/7.1

(57) ABSTRACT

An improved BRET assay, wherein the BRET signal is enhanced and/or prolonged. The improved BRET assay comprises the steps of i) adding a substrate to a cell comprising GPCR-Rluc fusion protein and a β-arrestin-GFP fusion protein, wherein the (β-arrestin is mutated, ii) adding a ligand to obtain, if possible, a GPCR-Rluc/(β-arrestin-GFP complex, and iii) measuring a BRET signal to obtain a BRET ratio, wherein the improvement leads to an increased BRET ratio compared with the ratios obtained by use of the same process employing a β-arrestin-GFP fusion protein wherein the β-arrestin is the wild type β-arrestin, or employing a 13-arrestin-GFP fusion protein, wherein the (β-arrestin is a β-arrestin specifically mutated so that it acts on the receptor independent of the receptors phosphorylation state. The invention further relates to a stable substrate solution for use in an improved BRET assay.

### FIELD OF THE INVENTION

The present invention relates to an improved BRET assay, wherein the BRET signal is enhanced and/or prolonged. The improved BRET assay comprises the steps of

i) adding a substrate to a cell comprising GPCR-Rluc fusion protein and a p-arrestin-GFP fusion protein, wherein the β-arrestin is mutated,

ii) adding a ligand to obtain, if possible, a GPCR-Rluc/β-arrestin-GFP complex, and

iii) measuring a BRET signal to obtain a BRET ratio.

The invention further relates to a stable substrate solution for use in an improved BRET assay.

### BACKGROUND OF THE INVENTION

BRET Assay

BRET (Bioluminescence Resonance Energy Transfer) assay is a protein-protein interaction assay. It is based on energy transfer from a bioluminescent donor to a fluorescent acceptor protein. This technology uses a Renilla luciferase (Rluc) as the donor and a Green Fluorescent Protein (GFP) as the acceptor molecule.

Rluc emits blue light (e.g. at 400 nm) in presence of its substrate. If a GFP molecule is in close proximity to the Rluc, it absorbs the blue light energy and re-emits green light (e.g. at 515 nm). The BRET signal, therefore, is measured by the amount of green light emitted by GFP as compared to the blue light emitted by Rluc. The ratio of green to blue increases as the two proteins are brought into proximity. BRET assays are performed by genetically fusing Rluc and GFP to biological partners that are expected to interact in a cell-based assay.

However, in certain situations where a GPCR-arrestin based assay is used, the BRET signal is relatively weak and short termed. Thus, there is a need for improving the BRET assay in order to obtain a prolonged and/or enhanced signal.

### DETAILED DISCLOSURE OF THE INVENTION

Accordingly, the present invention provides an improved BRET assay that comprises the following steps:

i) adding a substrate to a cell comprising GPCR-Rluc fusion protein and a β-arrestin-GFP fusion protein, wherein the β-arrestin is mutated,

ii) adding a ligand to obtain, if possible, a GPCR-Rluc/β-arrestin-GFP complex, and

iii) measuring a BRET signal to obtain a BRET ratio,

wherein the improvement leads to an increased BRET ratio compared with the ratios obtained by use of the same process employing a β arrestin-GFP fusion protein, wherein the β-arrestin is the wild type β-arrestin or employing a β-arrestin-GFP fusion protein, wherein the β-arrestin is a β-arrestin is specifically mutated so that it acts on the receptor independent of the receptors phosphorylation state.

G Protein-Coupled Receptors for Use in the Present Invention

The G protein-coupled receptors (GPCRs) constitute the largest family of proteins in the human genome and function as receivers of all kinds of chemical signals. The spectrum of hormones, neurotransmitters, paracrine mediators etc., which act through G-protein coupled receptors includes all kinds of chemical messengers: Ions (calcium ions acting on the parathyroid and kidney chemosensor), amino acids (glutamate and -amino butyric acid-GABA), monoamines (catecholamines, acetylcholine, serotonin, etc.), lipid messengers (prostaglandins, thromboxane, anandamide, (endogenous cannabinoid), platelet activating factor, etc.), purines (adenosine and ATP), neuropeptides (tachykinins, neuropeptide Y, endogenous opioids, cholecystokinin, vasoactive intestinal polypeptide (VIP), plus many others), peptide hormones (angiotensin, bradykinin, glucagon, calcitonin, parathyroid hormone, etc.), chemokines (interleukin-8, RANTES, MIP-1alpha etc.), glycoprotein hormones (TSH, LH/FSH, choriongonadotropin, etc.), as well as proteases (thrombin). In our sensory systems, G-protein coupled receptors are involved both as the light sensing molecules in the eye, i.e. rhodopsin and the color pigment proteins, and as several hundreds of distinct odorant receptors in the olfactory system as well as a large number of taste receptors. Structurally, G protein coupled receptors (GPCRs) are characterized by seven hydrophobic helical transmembrane segments connected by intra- and extracellular loops and are accordingly often referred to as 7TM receptors.

Examples of 7TM receptors are the receptors for (—in brachet the receptor subtypes are mentioned): acetylcholine (m1-5), adenosine (A1-3) and other purines and purimidines (P2U and P2Y1-12), adrenalin and noradrenalin (α1A-D, α2A-D and β1-3), amylin, adrenomedullin, anaphylatoxin chemotactic factor, angiotensin (AT1A, −1B and −2), apelin, bombesin, bradykinin (1 and 2), C3a, C5a, calcitonin, calcitonin gene related peptide, CD97, conopressin, corticotropin releasing factor (CRF1 and −2), calcium, cannabinoid (CB1 and −2), chemokines (CCR1-11, CXCR1-6, CX3CR and XCR), cholecystokinin (A-B), corticotropin-releasing factor (CRF1-2), dopamine (D1-5), eicosanoids, endothelin (A and B), fMLP, Frizzled (Fz1,2,4,5 and 7-9), GABA (B1 and B2), galanin, gastrin, gastric inhibitory peptide, glucagon, glucagon-like peptide I and II, glutamate (1-8), glycoprotein hormone (e.g. FSH, LSH, TSH, LH), growth hormone releasing hormone, growth hormone secretagogue/Ghrelin, histamine (H1-4), 5-hydroxytryptamine (5HT1A-1F, −2A-C and −4-7), leukotriene, lysophospholipid (EDG1-4), melanocortins (MC1-5), melanin concentrating hormone (MCH 1 and 2), melatonin (ML1A and 1B), motilin, neuromedin U, neuropeptide FF (NFF1 and 2), neuropeptide Y (NPY1,2,4,5 and 6), neurotensin (1 and 2), nocioceptin, odor components, opiods (κ, δ, μ and x), orexins(OX1 and −2), oxytocin, parathyroid hormone/parathyroid hormone-related peptides, pheromones, platelet-activating factor, prostaglandin (EP1-4 and F2) prostacyclin, pituitary adenylate activating peptide, retinal, secretin, smoothernd, somatostatins (SSTR1-5), tachykinins (NK1-3), thrombin and other proteases acting through 7TM receptor, thromboxane, thyrotropin-releasing hormone, vasopressin (V1A, −1B and −2), vasoactive intestinal peptide, urotensin II, and virally encoded receptors (US27, US28, UL33, UL78, ORF74, U12, U51); and 7TM proteins coded for in the human genome but for which no endogenous ligand has yet been assigned such as mas-proto-oncogene, EBI (I and II), lactrophilin, brain specific angiogenesis inhibitor (BAI1-3), EMR1, RDC1 receptor, GPR12 receptor or GPR3 receptor, and 7TM proteins coded for in the human genome but for which no endogenous ligand has yet been assigned.

Arrestins and GRKs Role in Receptor Signalling

Arrestins and GRKs (G-protein coupled receptor kinases) play important roles in the regulation of 7TM receptor responsiveness by terminating the G protein mediated signal. After agonist binding to a GPCR, the receptor changes conformation and is then phosphorylated by a GRK. Arrestins will then translocate to the phosphorylated receptor and bind to the receptor within seconds or minutes after agonist stimulation. Full inactivation of the G-protein mediated 7TM receptor signaling is achieved through binding of one of a family of arrestin molecules, which sterically hinder G protein binding.

Arrestin functions as an adaptor protein, which will connect the receptor to clathrin and AP-2 in clathrin coated pits, which results in sequestration of the receptor into intracellular vesicles of the endosomal pathway in which dynamin plays an important role in the actual vesicular sequestration process. The mechanisms involved in the transport of the arrestin-receptor complex to the clathrin coated pit is not fully understood, but it is becoming clear that the binding of arrestin to parts of the cell membrane e.g. to phosphoinositides is essential.

The family of arrestins has at least four members showing a high degree of amino acid homology and classified primarily on the basis of tissue distribution. They include (i) visual arrestin and (ii) C-arrestin, which are mostly restricted to the eye, and the non-visual-arrestins (iii) β-arrestin 1 and (iv) β-arrestin 2, distributed ubiquitously in almost every tissue. β-arrestins share more than 70% amino acid identity.

Arrestins are composed of three structural and functional parts, an amino-terminal domain, which binds to the receptor, a carboxyl-terminal domain, which connects to proteins involved in receptor-sequestration, such as clathrin and AP-2 (adaptor protein 2) and a central part which connects to components of the cell membrane, such as phosphoinositides. Visual arrestins, which mainly interacts with the rhodopsin receptor, are very weak in their clathrin-association and are in general not considered to be capable of mediating receptor internalisation.

As described above arrestin are translocated to the activated and phosphorylated GPCR within minutes after agonist simulation. This interaction is universal for almost all GPCRs upon activation. Thus, a BRET assay based on the GPCR-arrestin interaction, wherein the GPCR is fused with Rluc and arrestin is fused with GFP, is a very useful assay for a wide range of receptors, and it also provides means for the discovery of ligands that interact with GPCRs of unknown function i.e. orphan GPCRs.

However, as described above, arrestin functions as an adaptor protein, which will connect the receptor to clathrin and AP-2, which results in sequestration of the receptor into intracellular vesicles. After internalization of the receptor/arrestin complex, arrestin will dissociate from the receptor and the BRET signal is terminated. The dissociation kinetics can be fast or slow depending on the receptor type. For Class A type receptors, the dissociation is usually fast, whereas for Class B type receptors the dissociation is slower.

As mentioned above, β-arrestin associates to a receptor after the receptor has been phosphorylated by a GRK.

GRKs are 57-80 kDa proteins that are members of the large family of serine/threonine kinases. The human GRKs are part of a family of at least 7 GRKs (GRK1-7). Some of the GRKs have been shown to be expressed in a tissue specific manner, i.e. GRK1 (rhodopsin kinase), which is expressed in retina, and GRK4, which is expressed in testis. In contrast, the other GRKs are more widely distributed and evidence exists to suggest that these GRKs are likely to be involved in desensitization of multiple types of GPCRs.

Definitions

Throughout the text including the claims, the following terms shall be defined as indicated below.

In the present context the term “BRET ratio” is intended to mean the ratio of green light emitted by GFP as compared to the blue light emitted by Rluc. In the present context the terms “BRET signal” and “BRET ratio” are intended to have the same meaning.

A “ligand” is intended to include a substance that either inhibits or stimulates the activity of a receptor and/or that competes for the receptor in a binding assay. An “agonist” is defined as a ligand increasing the functional activity of a biological target molecule. An “antagonist” is defined as a ligand decreasing the functional activity of a biological target molecule either by inhibiting the action of an agonist or by its own intrinsic activity. An “inverse agonist” (also termed “negative antagonist”) is defined as a ligand decreasing the basal functional activity of a biological target molecule

In the present context the term “improved BRET assay” denotes an assay where the BRET ratio is increased by at least about 5% such as, e.g., at least about 10%, at least about 15%, at least about 20%, at least about 25%, at least about 30%, at least about 35%, at least about 40%, at least about 45%, at least about 50%, at least about 55%, at least about 65%, at least about 70%, at least about 75%, at least about 80%, or more.

In the present context, the term “β-arrestin that is specifically mutated so that it acts on the receptor independent of the receptors phosphorylation state” means that β-arrestin is mutated deliberately with the purpose of becoming phosphorylation independent. An example of such a mutation is R169E human β-arrestin-2, wherein arginine has been changed to glutamic acid. β-arrestins being mutated for other purposes, but accidentally also being phosphorylation independent, are not encompassed in this definition.

As described above, the present invention describes an improved BRET assay wherein the BRET signal is enhanced and/or prolonged.

Since the BRET signal is dependent on the association/dissociation of the GPCR-Rluc/β-arrestin-GFP complex, prevention of the dissociation of the complex will enhance and/or prolong the BRET signal.

Thus, the invention also provides an improved assay, wherein the separation of β-arrestin-GFP from GPCR-Rluc/β-arrestin-GFP complex is delayed and/or inhibited.

As described above, the GPCR-Rluc/β-arrestin-GFP complex dissociates when the complex is internalized. Thus, inhibition of the internalization will prevent dissociation and accordingly, the BRET signal will be enhanced and/or prolonged. Accordingly, the invention also relates to an improved assay wherein the internalization of GPCR-Rluc/β-arrestin-GFP complex is inhibited.

One way of inhibiting internalization is to prevent the binding of β-arrestin to clathrin and AP-2. In an improved assay according to the invention, β-arrestin is mutated so that its binding to clathrin and/or AP2 is impaired.

As described above, the carboxyl-terminal domain of arrestins connects to proteins involved in receptor-sequestration, such as clathrin and AP-2. Accordingly, in one assay according to the invention, the β-arrestin is truncated so that it does not contain any clathrin and/or AP2 binding sites, i.e. a part of the β-arrestin from the C-terminal end has been deleted.

In one assay according to the invention β-arrestin is mutated by deletion, insertion or substitution so that one or more AP2 binding sites of β-arrestin are impaired in their binding to AP2.

Specific examples of a truncated β-arrestin are a human β-arrestin-1 374 stop mutant or human β-arrestin-2 373 stop mutant.

A specific example of a β-arrestin mutated by substitution is the human β-arrestin-2 R393E; R395E mutant, wherein the amino acids number 393 and 395 have been changed from arginine to glutamic acid. Another example of a β-arrestin mutated by substitution is the human β-arrestin-2 R393A; R395A mutant, wherein the amino acids number 393 and 395 have been changed from arginine to alanine. Other substitutions may of course be used.

As described above, there are at least 4 family members of arrestins. Furthermore, the arrestins are found in animals including rodents, swine, poultry, cattle, sheep, goats, horses, cats, dogs, monkeys and humans. Thus, the specific mutations mentioned only tend as illustrative examples. All arrestins are usable in an assay according to the invention, and the specific position of the truncation and other mutations will depend on the species and type of arrestin. As an example, to impair the AP2 binding sites in bovine β-arrestin-2 amino acids number 394 and 396 should be substituted from arginine to glutamic acid as compared to the R393E and R395E substitutions in human β-arrestin.

Another way of inhibiting the internalization of the GPCR-Rluc/β-arrestin-GFP complex is to prevent the binding of β-arrestin to phosphoinositide. By impairing the binding of β-arrestin to phosphoinositide, the transport of the GPCR-Rluc/β-arrestin-GFP complex to the clathrin coated pits is impaired.

In order to impair binding to phosphoinositide β-arrestin may be mutated in any suitable way e.g. by deletion, insertion or substitution.

A specific example of a β-arrestin mutant, wherein the binding to phosphoinositide is impaired is the triple mutant human β-arrestin-2 K233Q; R237Q; K251Q, wherein amino acid no. 233 has been changed from lysine to glutamine, amino acid no. 237 has been changed from arginine to glutamine and amino acid no. 251 has been changed from lysine to glutamine.

The examples given above are all related to ways of improving/prolonging the BRET signal by prevention of the dissociation of the GPCR-Rluc/β-arrestin-GFP complex

The invention also provides an improved assay, wherein the association of β-arrestin-GFP to the GPCR is increased, which will in turn increase the BRET signal.

As described above, β-arrestin associates to a receptor after the receptor has been phosphorylated by a GRK. In some cases the receptor used in an assay as described herein will be phosphorylated slowly and/or not fully, i.e. if the amount of GRK naturally present in the cells is low. This results in slow and/or reduced binding of β-arrestin to the receptor, leading to a reduced BRET signal. Thus, by increasing the phosphorylation of the receptor the BRET signal will be enhanced. As the phosphorylation of a GPCR by a GRK is a virtually universal event, a GPCR-Rluc/β-arrestin-GFP based assay as described above, further aided by the addition of a GRK is a very useful assay, especially if the phosphorylation of the GPCR is rate limiting step in the association of GPCR-Rluc and β-arrestin-GFP.

Accordingly, the invention also relates to an improved assay, wherein the cell comprises a further amount of G-protein coupled receptor kinase as compared to the amount of GRK naturally present in the cell.

In a specific example, the G-protein coupled receptor kinase is GRK 2 or GRK-5.

As described above, there are at least 7 family members of human GRKs. Furthermore, the GRKs are found in other animals including rodents, swine, poultry, cattle, sheep, goats, horses, cats, dogs and monkeys. Thus, the specific GRKs mentioned only tend as illustrative examples as all GRKs are usable in an assay according to the invention.

The β-arrestin may be further mutated so that it, besides having impaired binding capability to clathrin, AP2 and/or phosphoinositide, also is phosphorylation independent. As described above, the binding of β-arrestin to the GPCR requires the phosphorylation of the receptor, the latter being a rate limiting step in some cases. By using a phosphorylation independent β-arrestin mutant in an assay according to the invention, the degree of phosphorylation of the receptor will no longer have any influence on the formation of the GPCR-Rluc/β-arrestin-GFP complex, leading to a higher BRET signal in cases, where phosphorylation of the receptor is a rate limiting step.

The invention also relates to all suitable combinations of β-arrestin mutants, i.e, a mutant having impaired binding capability to both clathrin and/or AP2 and phosphoinositide.

### ASPECTS OF THE INVENTION

The improved BRET assay may be used in drug discovery methods, such as screening assays for identifying new ligands of GCPRs. The BRET assay may also be used for the discovery of ligands that interact with GPCRs of unknown function i.e. orphan GPCRs.

The ligands may be agonists or antagonists. If the ligand is a known antagonist, or if the assay is set up to screen for unknown antagonists, the improved BRET assay further comprises the addition of an agonist after adding the antagonist, or suspected antagonist ligand.

The invention also relates to an improved assay according to the invention for use in high-throughput screening.

One of the problems associated with setting up an assay, such as, e.g. a high through-put screening (HTS) assay, is to prepare a suitable solution of the substrate, DeepBlueC™. If the stock solution of DeepBlueC™ is diluted to the relevant concentration for use in a BRET assay in D-PBS as recommended, it will form a precipitate within 10 to 20 minutes, making it virtually impossible to use for a HTS assay.

The present inventors have found that a solution of DeepBlueC™ in a proper amount of organic solvent prevents the formation of precipitate. Accordingly, the present invention relates to a solution comprising DeepBlueC™ and one or more organic solvents, wherein no visual precipitate is formed after storage at room temperature for at least 30 min such as, e.g., at least about 45 min, for at least about 1 hr, for at least about 1.5 hrs, for at least about 2 hrs, for at least about 2.5 hrs, for at least about 3 hrs, for at least about 3.5 hrs or for at least about 4 hrs.

The organic solvent may be chosen from any organic solvent usable in a BRET assay, such as, e.g. an alkanol including ethanol, propanol, isopropanol, and butanol. In a specific embodiment, the solvent is ethanol.

As illustrated in the examples, the formation of precipitate is interrelated to the concentration of the organic solvent in the solution.

When the concentration of organic solvent is decreased from a optimal level, the amount of precipitate will increase, and the time before formation of precipitate will decrease. On the other hand, if the concentration of organic solvent in the solution is too high it may not be suitable for use in a BRET assay, as the BRET signal will decrease if the percentage of organic solvent in the assay gets too high.

The invention relates to a solution, comprising from about 15% v/v EtOH to about 100 v/v % EtOH, such as, e.g. from about 20% v/v EtOH to about 90% v/v EtOH, from about 30% v/v EtOH to about 80% v/v EtOH, from about 35% v/v EtOH to about 75% v/v EtOH, from about 35% v/v EtOH to about 70% v/v EtOH, from about 35% v/v EtOH to about 65% v/v EtOH, from about 35% v/v EtOH to about 60% v/v EtOH, from about 40% v/v EtOH to about 60% v/v EtOH, from about 40% v/v EtOH to about 55% v/v EtOH or from about 40% v/v EtOH to about 50% v/v EtOH.

In a specific embodiment, the invention relates to a solution, comprising DeepBlueC™ in 40% v/v EtOH.

The invention also relates to a solution, further comprising D-PBS.

The invention also relates to a method for preparing a solution as described above, the method comprising diluting a stock solution of DeepBlueC™ in a solution comprising one or more organic solvents. The actual dilution of DeepBlueC™ depends on the conditions of the specific assay, wherein the solution is to be used. A specific example of a suitable solution appears from the Examples, without limiting the invention hereto.

The substrate solution is suitable for use in an improved BRET assay as described herein. I.e., the invention relates to an improved assay according to any of the preceding claims, wherein the substrate is used in the form of a solution from which no visual precipitate is formed after storage at room temperature for at least 30 min such as, e.g., for at least about 45 min, for at least about 1 hr, for at least about 1.5 hrs, for at least about 2 hrs, for at least about 2.5 hrs, for at least about 3 hrs, for at least about 3.5 hrs or for at least about 4 hrs.

The invention also relates to an improved assay, wherein the solution containing the substrate comprises one or more organic solvents. The one or more organic solvents may be selected from alkanols including ethanol, propanol, isopropanol, and butanol. In a specific embodiment, the solvent is EtOH.

The invention further relates to an improved assay, wherein the solution comprises from about 15% v/v EtOH to about 100 v/v % EtOH, such as, e.g. from about 20% v/v EtOH to about 90% v/v EtOH, from about 30% v/v EtOH to about 80% v/v EtOH, from about 35% v/v EtOH to about 75% v/v EtOH, from about 35% v/v EtOH to about 70% v/v EtOH, from about 35% v/v EtOH to about 65% v/v EtOH, from about 35% v/v EtOH to about 60% v/v EtOH, from about 40% v/v EtOH to about 60% v/v EtOH, from about 40% v/v EtOH to about 55% v/v EtOH or from about 40% v/v EtOH to about 50% v/v EtOH.

In a specific embodiment, the solution comprises DeepBlueC™ in 40% v/v EtOH.

### OTHER ASPECTS OF THE INVENTION

Other aspects of the invention appear from the appended claims. The details and particulars described above and relating to the method according to the invention apply mutatis mutandis to the other aspects of the invention.

### LEGENDS TO FIGURES

FIG. 1 shows internalization of the NK1 and the β2AR receptors co-expressed in cells together with WT or one of the three different β-arrestin mutants: human β-arrestin-2 R393E; R395E mutant, human β-arrestin-2 373 stop mutant or human β-arrestin-2 R169E mutant.

FIG. 2 shows the specific BRET ratio of the NK1 and the β2AR receptors co-expressed with WT or one of the three different β-arrestin mutants: human β-arrestin-2 R393E; R395E mutant, human β-arrestin-2 373 stop mutant or human β-arrestin-2 R169E mutant.

FIG. 3 shows the light emission from renilla luciferase after addition of DeepBlueC dissolved in buffer (D-PBS with 1000 mg/l L-Glucose), buffer with 6% ethanol or buffer with 40% ethanol.

The following examples are intended to illustrate the invention without limiting it thereto.

### EXAMPLES

NK-1 Receptor Internalization Assays

COS7 cells in 75 cm² flask (3×10⁶ cells/flask) were used for transfection. NK-1/Rluc receptor (2 μg cDNA/flask) was coexpressed together with 6 μg GFP/β-arrestin 2, 6 μg GFP²/β-arrestin R169E, 6 μg GFP²/β-arrestin Lys 373 stop or 6 μg GFP²/β-arrestin R393E, R395E. At the end of transfection period (3-5 hours), cells were washed twice with PBS, trypsinased and plated at a density of 2.5×10⁵ cells per well in 12-well plates. After 48 hours, cells were washed once with assay medium (HEPES-modified DMEM with 0.1% BSA, pH 7.4) and incubated in assay medium for at least 1 hour before being incubated with ¹²⁵I-labeled SP (30000 cpm/well) in 0.5 ml assay medium 10 min at 37 C. Cells were then transferred onto ice and washed twice with ice-cold PBS. Subsequently, the extracellular receptor-associated ligand was removed by washing once with 1 ml of acid solution (50 mM acetic acid and 150 mM NaCl, pH 2.8) for 12 min. The acid wash was collected to determine the surface-bound radioactivity, and the internalized radioactivity was determined after solubilizing the cells in 0.2 M NaOH and 1% SDS (NaOH/SDS) solution. Nonspecific binding for each time point was determined under the same conditions in the presence of 1 μM unlabeled agonist (SP). After subtraction of nonspecific binding, the internalized radioactivity was expressed as a percentage of the total binding.

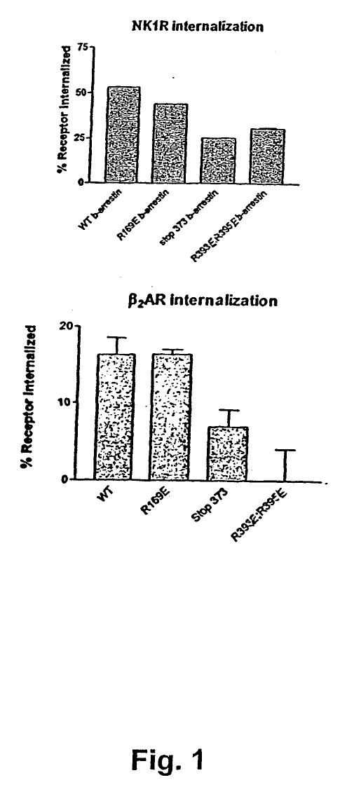

FIG. 1 shows the internalization of the NK1-R co-expressed in cells together with WT or one of the three different β-arrestin mutants. The figure illustrates that the human β-arrestin-2 R393E; R395E mutant and the human β-arrestin-2 373 stop mutant are inhibiting the internalization.

β2AR Internalization Assays

COS-7 cells in 75 cm² flask (3×10⁶ cells/flask) were used for trasfection. β₂AR/Rluc receptor (1.3 μg cDNA/flask) was coexpressed together with 6.5 μg GFP²/β-arrestin 2, 6.5 μg GFP²/β-arrestin R169E, 6.5 μg GFP²/β-arrestin Lys 373 stop or 6.5 μg GFP²/β-arrestin R393E, R395E. Receptor internalization assay was based on protocol described by Barak and Caron J Recept Signal Transduct Res 1995 January-March;15(14):677-90. At the end of transfection period (3-5 hours), cells were washed twice with PBS, trypsinesed and plated at a density of 2.5×10⁵ cells per well in 12-well plates. After 48 hours, cells were washed once with assay medium (HEPES-modified DMEM with 0.1% BSA, pH 7.4) and serum-starved in the same medium for additional 2-3 hours before being stimulated with 1 mM isopterenol for 10 min at 37° C. Stimulation was stopped by washing the cells with ice-cold PBS. Cells were then subjected to [¹²⁵I]-pindolol binding at 4° C. for 3 h and the fraction of internalized receptors determined relative to unstimulated cells. Non-specific binding was determined under the same conditions in the presence of 1 μM pindolol.

FIG. 1 shows the internalization of the β2AR co-expressed in cells together with Wt or one of the three different β-arrestin mutants. The figure illustrates that the human β-arrestin-2 R393E; R395E mutant and the human β-arrestin-2 373 stop mutant are inhibiting the internalization.

It is also shown that the effect is most significant for the β2AR receptor as compared to the NK1 receptor.

BRET Assay

Cell Preparation for BRET Assay:
1. 48 hrs after transfection with 2 μg receptor-Rluc DNA (e.g. NK1-Rluc or β2AR-Rluc) and 5 μg β-arrestin-2 DNA (e.g. the R393E; R395E mutant) (for assays with GRK also 2 μg GRK DNA, e.g. GRK-5) media was removed and cell washed 1× with PBS
2. 1 ml 1× trypsin (T75 flask) was added and incubated 3-5 min at 37 C
3. 10 mL complete media was added.
4. Cells were transferred to tube and spun down (5 min, 800 rpm)
5. Media was removed, and cells resuspended in 10 ml PBS and counted, spun down (5 min, 800 rpm)
6. Resuspended in D-PBS with 1000 mg/l L-Glucose (#14287) to a density of 2×10⁶ cells/ml
7. Cells were left at room temperature for at least 30 min, to stabilize readings.

Assay:
1. Dilution of DeepBlueC to 100 μM in 40% ethanol in D-PBS with 1000 mg/l L-Glucose (#14287) (light sensitive!!). The DeepBlueC™ stock solution comprises 1 mM DeepBlueC™ in 100% v/v EtOH.

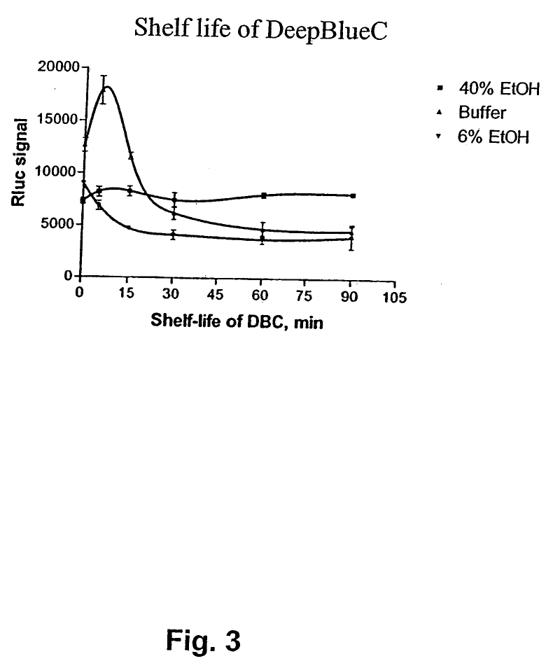

The dilution of DeepBlueC in 40% ethanol/buffer is essential for the stability of DeepBlueC. If DeepBlueC is diluted in buffer alone, it will precipitate within 10 to 20 minutes, making it virtually impossible to use the assay in HTS mode. However, when diluted in 40% ethanol DeepBlueC will be stable for hours. FIG. 3 shows the light emission from renilla luciferase after addition of DeepBlueC dissolved in buffer (D-PBS with 1000 mg/l L-Glucose), buffer with 6% ethanol or buffer with 40% ethanol. It can be seen that the signal from the experiment using 40% ethanol is stable for the duration of the experiment. On the other hand the signals from experiments using 6% or 0% ethanol rapidly decreases.
2. 100 μl of resuspended cells were transferred into wells in 96-well white optiplate plate
3. 12 μl agonist was added.
4. 5 μl of diluted DeepBlueC/well was added, and the plate was read.

Antagonist:
1. Dilution of DeepBlueC to 100 μM in 40% ethanol in D-PBS with 1000 mg/l L-Glucose (#14287) (light sensitive!!)
2. 100 μl of resuspended cells was transferred into wells in 96-well white optiplate plate
3. 14 μl antagonist was added (wait 5 min.)
4. 14 μl agonist was added.
5. 6 μl of diluted DeepBlueC/well was added, and the plate read.

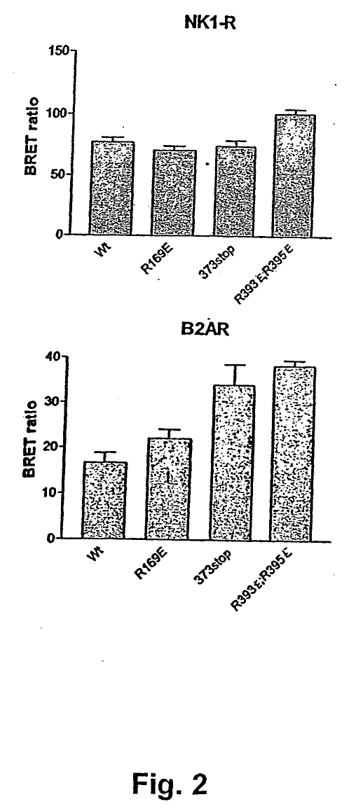

FIG. 2 shows the specific BRET ratio of the NK1 and the β2AR receptors coexpressed with WT and the three different P-arrestin mutants: human β-arrestin-2 R393E; R395E mutant, human β-arrestin-2 373 stop mutant or human β-arrestin-2 R169E mutant. It is seen that the human β-arrestin-2 R393E; R395E mutant and the human β-arrestin-2 373 stop mutant are increasing the BRET signal significantly for the β2AR receptor, whereas the effect is less pronounced for the NK1 receptor. The observed results are expected since the NK1 receptor is a class B receptor and the β2AR receptor is a class A receptor.

1-35. (canceled)

36. An assay comprising
i) adding a substrate to a cell comprising GPCR-Rluc fusion protein and a β-arrestin-GFP fusion protein, wherein the β arrestin is mutated,
ii) adding a ligand to the admixture of i), and
iii) measuring a BRET signal to obtain a BRET ratio,
wherein an increased BRET ratio is provided compared with the ratios obtained by use of the same process employing a β-arrestin-GFP fusion protein wherein the β-arrestin is the wild type β-arrestin, or employing a β-arrestin-GFP fusion protein, wherein the β-arrestin is a β-arrestin specifically mutated so that it acts on the receptor independent of the receptors phosphorylation state.

37. The assay of claim 36 wherein the ligand addition can provide a GPCR-Rluc/β-arrestin-GFP complex.

38. The assay of claim 37 wherein separation of β-arrestin-GFP from GPCR-Rluc/β-arrestin-GFP complex is delayed and/or inhibited.

39. The assay of claim 37 wherein internalization of the GPCRRluc/β-arrestin-GFP complex is inhibited.

40. The assay of claim 36 wherein β-arrestin is mutated so that its binding to clathrin and/or AP2 is impaired.

41. The assay of claim 36 wherein β-arrestin is truncated so that it does not contain any clathrin and/or AP2 binding sites.

42. The assay of claim 36 wherein β-arrestin is mutated by deletion, insertion or substitution so that one or more AP2 binding sites are impaired in their binding to AP2.

43. The assay of claim 36 wherein β-arrestin is mutated so that its binding to phosphoinositide is impaired.

44. The assay of claim 36 wherein the cell comprises a further amount of G-protein coupled receptor kinase (GRK) as compared to the amount of GRK naturally present in the cell.

45. The assay of claim 44 wherein the G-protein coupled receptor kinase is GRK 2.

46. The assay of claim 44 wherein the G-protein coupled receptor kinase is GRK 5.

47. The assay of claim 36 wherein β-arrestin is further mutated so that it is phosphorylation independent.

48. The assay of claim 36 wherein β-arrestin is originating from an animal source.

49. The assay of claim 36 wherein β-arrestin is a β-arrestin-1 or β-arrestin-2.

50. The assay of claim 36 wherein the β-arrestin is a human β-arrestin-1 374 stop mutant or human β-arrestin-2 373 stop mutant.

51. The assay of claim 36 wherein the β-arrestin is a human β-arrestin-2 R393E; R395E mutant.

52. The assay of claim 36 wherein the β-arrestin is a human β-arrestin-2 R393A; R395A mutant.

53. The assay of claim 36 wherein the β-arrestin is human β-arrestin-2 K233Q; R237Q; K251Q mutant.

54. The assay of claim 36 for use in drug discovery methods.

55. The assay of claim 36 for use in high-throughput screening:

56. The assay of claim 36 wherein the substrate is DeepBlueC™.

57. The assay of claim 36 wherein the substrate is used in the form of a solution from which no visual precipitate is formed after storage at room temperature for at least 30 minutes.

58. The assay of claim 57 wherein the solution comprising the substrate comprises one or more organic solvents.

59. The assay of claim 58 wherein the one or more organic solvents are selected from alkanols including ethanol, propanol, isopropanol, and butanol.

60. The assay of claim 58 wherein the solvent is EtOH.

61. The assay of claim 58 wherein the solution comprises from about 15% v/v EtOH to about 100 v/v % EtOH.

62. The assay of claim 61 wherein the solution comprises DeepBlueC™ in 40% v/v EtOH.

63. A solution comprising DeepBlueC™ and one or more organic solvents, wherein no visual precipitate is formed after storage at room temperature for at least 30 minutes.

64. A solution of claim 63 wherein the one or more organic solvents are selected from alkanols including ethanol, propanol, isopropanol, and butanol.

65. A solution of claim 63 wherein the solvent is EtOH.

66. A solution of claim 65 comprising from about 15% v/v EtOH to about 100 v/v % EtOH.

67. A solution according to claim 66 comprising DeepBlueC™ in 40% v/v EtOH.

68. A method for preparing a solution of claim 63, the method comprising: diluting a stock solution of DeepBlueC™ in a solution comprising one or more organic solvents.

69. The assay of claim 36 wherein a GPCR ligand is identified.

70. The assay of claim 69 wherein the ligand is an agonist.

71. The assay of claim 69 wherein the ligand is an antagonist.
